(19) United States
(12) Patent Application Publication
(10) Pub. No.: US 2023/0218412 A1
(43) Pub. Date: Jul. 13, 2023

(54) PROSTHETIC DEVICES AND METHODS OF MANUFACTURING THE SAME

(71) Applicant: Human Plant Solutions, Newton, KS (US)

(72) Inventor: Kyle Edward Trivisonno, Newton, KS (US)

(21) Appl. No.: 18/183,219

(22) Filed: Mar. 14, 2023

Related U.S. Application Data
(63) Continuation of application No. 16/365,200, filed on Mar. 26, 2019.
(60) Provisional application No. 62/648,018, filed on Mar. 26, 2018.

(51) Int. Cl. A61F 2/50; A61F 2/80; A61F 2/60; A61F 2/54

(52) U.S. Cl. CPC A61F 2/5044; A61F 2/80; A61F 2/60; A61F 2/54; A61F 2002/5055; A61L 27/3637

(57) ABSTRACT

Embodiments of the present invention provide improved prosthetic devices. The prosthetic devices may be fabricated using environmental friendly, renewable and sustainable materials. Methods of manufacturing the devices are also provided. Additionally, the present invention provides an environmental friendly, renewable and sustainable substitute for carbon and/or fiberglass materials.

### RELATED APPLICATION DATA

The present application is a continuation of U.S. application Ser. No. 16/365,200, filed Mar. 26, 2029, which claims priority from and the benefit of U.S. Provisional Patent Application No. 62/648,018, filed Mar. 26, 2018, the disclosures of which are hereby incorporated herein by reference in their entireties.

### FIELD

The present invention is directed to an improved prosthetic device and method of manufacture. More particularly, the present invention is directed to products and prosthetic devices including natural materials and/or fibers and methods of manufacturing the same.

### BACKGROUND

Current industry standards for manufacturing definitive laminated prosthetic and orthotic devices generally employ materials including carbon fiber braids, fiberglass, synthetic man-made materials, or a combination thereof. These materials may be layered over a positive model of an intended prosthetic or orthotic device and held together by a resin.

These manufacturing techniques typically use a polyvinyl acetate barrier i.e., a polyvinyl acetate (PVA) bag that may be pulled over the positive model and sealed to a source of vacuum. This may create a vacuum tight barrier for a resin when added later to combine the layers of materials over the positive model. The number of layers of carbon fiber and like materials and reinforcement may be determined by a prosthetist or orthotist and may vary by weight and activity level of patient. Typically, in a prosthetic device these layers of material are tied into an attachment point so the “socket” may be attached to the subsequent parts of the prosthesis. (i.e., endo-skeletal components knee or foot). Accordingly, once a sufficient number of layers of carbon, fiberglass, etc. are disposed over the positive model a second PVA bag may be pulled over the layers to form a bag like structure. One end of the bag may be attached to a vacuum source, and the top of the bag may be left open for a resin to be poured through. The first and second PVA bags will create a barrier between the positive model, the layers of material that will result in the prosthetic device, and outside air. The vacuum source may then be turned on and the resin poured in the area filled with layers between the first and second PVA bags, until the layers of material is sufficiently saturated with resin and substantially no air may be left between the two PVA bags. After the resin hardens and cures the device is cut out and removed from the positive model. The trim line edges are then determined and finished to a smooth surface so there is no irritation to patient when worn. The device is then fit to a patient by a practitioner.

However, some issues that challenge prosthetic manufacturers as known to those skilled in the art include durability, residual-limb volume fluctuations, temperature and moisture control, and skin health among other issues. Further, the materials used for making the devices may not be environmentally friendly, and thus, may present issues when the time arises to dispose of these devices.

Accordingly, there is a need for an improved prosthetic device and method of manufacture that is capable of providing durable, comfortable, cost-effective, non-irritating and/or environmentally friendly prosthetic devices.

### SUMMARY

According to embodiments of the present invention, improved prosthetic devices and methods of manufacturing the same are provided. Exemplary prosthetic devices may be fabricated using environmentally friendly, renewable and/or sustainable materials. For example, the materials may include, but not be limited to, natural fibers such as hemp and other similar natural fibers.

By employing the disclosed improved prosthetic devices and methods of manufacture, prothesis may be designed and built to provide durable, comfortable, cost-effective, and/or environmentally friendly prosthetic devices, and the novel method of manufacture may assist in reducing the weight of the prosthetic device creating an ultralight definitive prosthetic device with equal or better tensile and/or integral strength compared to the prosthetic devices currently available on the market.

Embodiments of the present invention further provide materials and methods to replace traditional carbon fiber and/or fiberglass materials. The environmentally friendly, renewable and/or sustainable materials of the present invention, which may include, but not be limited to, natural fibers like hemp and other similar natural fibers, which may be used in materials used in construction and/or building materials, automotives, aircrafts, boats, bicycles (unicycles, tricycles, tandem bicycles, etc.), motorcycles, scooters, hoverboards, and other recreational vehicles, sports equipment, recreational equipment, musical equipment, electronics, ballistics, armor plating and various consumer products, and accessories associated with each.

### BRIEF DESCRIPTION OF DRAWINGS

The present invention is illustrated by way of example, and not by way of limitation, in the figures of the accompanying drawings.

FIG. 1 illustrates an environmental perspective of the prosthetic device in accordance with embodiments of the present disclosure.

FIGS. 2 through 8 are drawings illustrating stages in the method of manufacture of a prosthetic device in accordance with embodiments of the present disclosure.

FIG. 9 is a drawing illustrating a socket of a prosthetic device in accordance with embodiments of the present disclosure.

FIG. 10 is a drawing illustrating the prosthetic device in accordance with embodiments of the present disclosure.

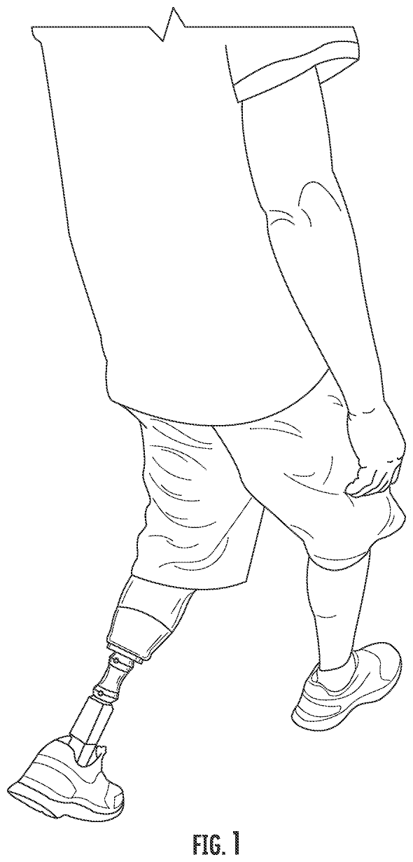

Unless otherwise indicated, illustrations in the figures are not necessarily drawn to scale.

### DETAILED DESCRIPTION

The foregoing and other aspects of the present invention will now be described in more detail with respect to other embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention pertains. The terminology used in the description of the invention herein is for the purpose of describing particular embodiments only and is not intended to be limiting of the invention. As used in the description of the invention and the claims set forth herein, the singular forms “a,” “an,” and “the” are intended to include the plural forms as well, unless the context clearly indicates otherwise. As used herein, the term “and/or” includes any and all combinations of one or more of the associated listed items.

All publications, patent applications, patents and other references cited herein are incorporated by reference in their entireties for the teachings relevant to the sentence and/or paragraph in which the reference is presented.

Embodiments of the invention as disclosed herein provide a prosthetic device and method of manufacture. More particularly, the present invention is directed to providing an improved prosthetic device including a natural material and methods of manufacture of the same. As used herein, “prosthetic” refers to a mechanical device that replaces a missing body part. As used herein “orthotic” means a device that is applied externally to a part of the body to support a part of the body, correct a deformity, relieve pain, and/or improve the function of a part of the body. Prosthetic and orthotic may be used interchangeably.

In one embodiment, the present disclosure aims on replacing the industry standard of carbon fiber, fiberglass, or synthetic fiber, and in particular, for fabrication of prosthetic and orthotic devices with natural materials as the source of strength of lamination. “Natural” materials or fibers refer to those derived and/or produced by plants and animals. As used herein, “natural” materials do not include carbon fiber or fiberglass fibers. The disclosure provides a method of using renewable and sustainable materials for fabrication in orthotics and prosthetics. Further, through the use of plant-based or animal-based, natural resources in the device, the workplace may likewise become a healthier environment for technicians and practitioners. In various embodiments, hemp and other like natural fibers may replace carbon, fiberglass and other hazardous and non-renewable materials in the layering or lay-up of materials used in a laminated prosthetic socket or orthotic devices either partially or completely. Furthermore, the bioavailability of hemp and natural fibers may render the natural fibers a lower-cost alternative compared to current standard products (such as carbon, fiberglass, and traditional fabrication materials) used in the industry as well as presents an environmentally friendly option for disposal.

As used herein, “hemp” also refers to “industrial hemp” and generally embraces any fibers derived from plants of the genus Cannabis. Bast fibers provide strength to the plant. The term “bast fibers” generally refers to the fibers that grow on the outside of the woody interior of the plant's stalk and under the bark. In particular embodiments, hemp fibers are derived from plants of the Cannabis genus. In further embodiments, the hemp fibers are derived from the plant species Cannabis sativa L., and in still further embodiments, Cannabis sativa. In further embodiments, fibers utilized in the prosthetic device are bast fibers, and in particular, bast fibers of hemp.

Accordingly, embodiments of the present inventive concept provide an improved prosthetic device. The prosthetic device may be fabricated using environmental friendly, renewable and sustainable materials. “Environmentally safe”, “environmentally preferred”, “environmentally friendly”, or “green” (which may be used interchangeably), as used herein refer to the quality of not being harmful to plants and/or animals when exposure is under normal conditions or deemed to inflict reduced, minimal, or no harm upon ecosystems and/or the environment as well as products that have a lesser or reduced effect on human health and the environment when compared with competing products or services that serve the same purpose

In some embodiments, the prosthetic device of any preceding claim, wherein the socket configured to accept the limb residuum includes at least 50%, 60%, 70%, 80%, 90%, 95% or 99% of an environmentally friendly, renewable and/or sustainable material including the natural materials or fibers described herein.

These materials may include, but not be limited to, natural materials or fibers such as hemp, flax, wood pulp, bamboo, tencel, cotton, wool, camel hair, cashmere, mohair, silk, jute, ramie and sisal. The natural fibers used herein may be engineered in a radial braided design and layered. This technique may assist in reducing the weight of the prosthetic device creating an ultralight definitive prosthetic device with equal or better tensile and integral strength compared to the prosthetic devices currently available in the market.

In one embodiment, the present invention provides a prosthetic device comprising an elongated element having an upper section and a lower section, the elongated element being fabricated of at least one layer formed of a natural material. The prosthetic device may possess substantial tensile strength, integral strength and be capable of storing and releasing energy.

In other embodiments, the present invention provides a prosthetic device comprising a socket configured to accept a limb residuum and comprising a resin composition or matrix reinforced by at least one of a natural material or fiber such as a plant and/or animal-based fiber; and a shank mechanically attached to the socket.

According to other embodiments, the present invention provides a socket configured to accept a limb residuum and comprising a resin composition or matrix reinforced by at least one of a natural material or fiber such as a plant and/or animal-based fiber.

In particular embodiments, the composition of matter including the natural material may comprise, consist or consist essentially of the natural material.

In some embodiments, the natural material is (a) braided to form a braided sleeve; (b) layered in a manner resulting in a bi-directionally woven strip of natural material; and/or (c) layered in a manner resulting in a radial braided natural material sleeve. In particular, manufacturing of the prosthetic device disclosed herein may include layering of natural materials to either partially or completely replace conventional materials like carbon fiber, and thus, the prosthetic device may include the natural materials described herein along with a carbon fiber material. The prosthetic device may also use a natural or plant-based resin to bind the various layers of natural fiber in the lamination formed by layering of the natural fibers.

In one embodiment, the prosthetic device may include manufacturing a first layer using hemp and/or other natural material to form a lightweight fiber braided sleeve. The light weight fiber braided sleeve may be used for the production of prosthetic limbs, braces, and a variety of other products that may be of need in the prosthetic and orthotic field.

In the layering process, the natural materials may be layered in a manner resulting in a bi-directionally woven strip of hemp and/or other natural fibers to reinforce the structural weak points within the prosthetic device i.e., socket, and provide extra support in areas of need. In a further step, in the layering process the natural materials may be layered in a manner resulting in a radial braided natural material sleeve to form a thicker component of structural support for a laminated product, i.e., the prosthetic device socket. In yet a further step, the woven materials formed in the previous steps may be strategically layered over a positive model under vacuum and then a resin poured over the layers to saturate them to create a definitive device as described herein above with reference to methods for currently available prosthetic devices made using carbon fibers and like materials. One of skill in the art may appreciate that in various embodiments, the number and positioning of each of the layers described in the various steps above may be combined and further depend on a practitioners' discretion and a user's needs. That is, in certain embodiments, each of what may be deemed three independent layers described in three steps may be used individually, in combination with each other, or in combination with traditional manufacturing materials like carbon fibers depending on the practitioners' discretion and a user's needs.

The resin used to pour over the layers of natural fiber include those resins known to those skilled in the art and used in the manufacture of prosthetic devices. Such resin materials include plant, acrylic or epoxy resins and combinations thereof. In particular, the resin may include, but are not limited to, rosin, damar, copal, sandarac, amber, manila, polyetherimide, polyethylene, polypropylene, polycarbonate, polyvinylchloride, polystyrene, epoxy resin, polytetrafluoroethylene, polyacetal, polyamide, polyurethane, ethylene-vinylacetate copolymer, polymethylmethacrylate, polyvinylalcohol, linear low density poly ethylene, low density polyethylene, high density polyethylene, acrylonitrile-butadiene-styrene, styrene-acrylonitrile, polyacrylonitrile, polybutadiene, polyacrylic acid, polyacrylimide, polysulfone, polyacetal, polyamide-imide, polytetrafluoroethylene, polyneoprene, polydimethylsiloxane, methyl-methacrylate, polymethylmethacrylate, polyetheretherketone, polyphenylenesulfide, polyvinylfluoride, polyvinylacetate, polyvinylidinefluoride, polyether sulfone, polycaprolactone and a copolymer thereof; a silicon resin; a natural rubber; a synthetic rubber; and a mixture thereof, acrylonitrile butadiene styrene (ABS), acrylic/PVC thermoplastic sheets, polycarbonate, co-polyester sheets, thermoplastic elastomer (TPE), polyvinyl chloride (PVC), polyolefin, polypropylene, isotatic polypropylene, polystyrene, homopolymer polypropylene, co-polymer polypropylene, polyethylene, and other suitable materials. In some embodiments, the resin is a modified acrylic resin, an epoxy acrylic resin or a polyester.

A resin may be poured into the prosthetic device being made, where natural fiber layers (for example, hemp layers) have already been assembled.

One skilled in the art may appreciate that a natural fiber, such as a hemp fiber (for example, in the form of a braided sleeve) may have an adequate amount of stretch within the material and may be manufactured to fit a wide variety of different sizes to best fit the practitioners' discretion and user's needs. In one embodiment, the fiber braided sleeve may be manufactured in different sized options measured by flat width for a practitioner or a company using the sleeve to manufacture prosthetic devices. In one embodiment, the braided sleeve may be formed using hemp exclusively, wherein the hemp may be braided substantially loosely so that the resultant braided sleeve may be reflected over various sized and shaped positive models for fabrication.

In one embodiment, the lightweight sleeve is woven using hemp and/or other materials as described herein to stretch over a positive model and provide a layer between the hemp braid for strength in the final product. The sleeve would have a range of sizes from which the practitioner may choose, which may also be measured by flat width. The hemp fiber would provide layers between the braid and other structural reinforcement to create a stronger final product. The hemp fiber may be blended with other materials to give it a stretchable characteristic that would allow it to be used on various sized models and contour around different substrates that need prosthetic or like devices fitted on them.

In one embodiment, the bi-directional woven reinforcement strips may be manufactured into rolls that may easily be cut into segments at the practitioners' discernment. In another embodiment, the strips may include an adhesive backing for application to the positive model for the reinforcement of weak spots.

In yet another embodiment, the resin employed to join the various layers of the laminate may include a natural material such as a plant-based resin that may be manufactured using hemp and/or other natural and sustainable resources. In certain embodiments, the resin may be a synthetic bio-degradable resin.

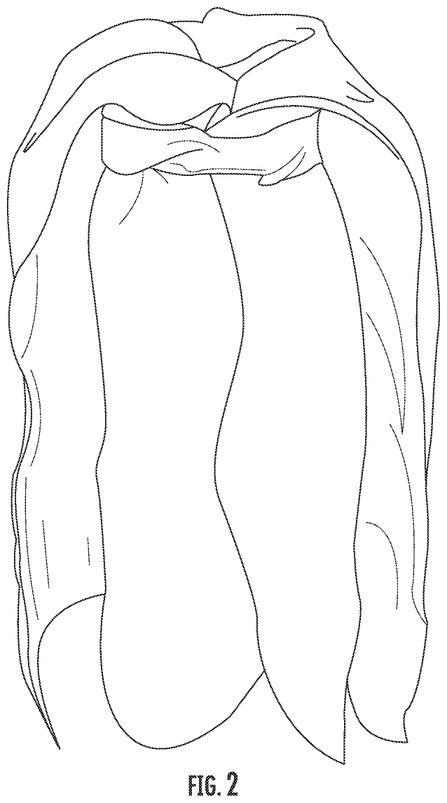

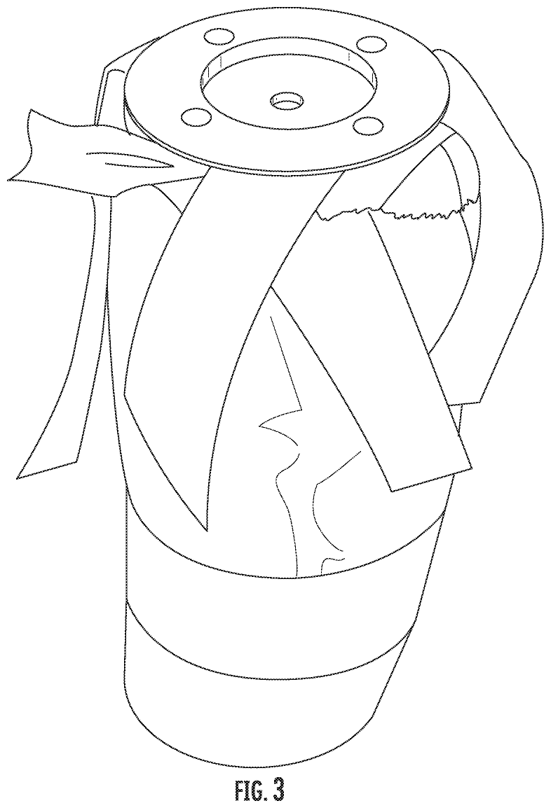

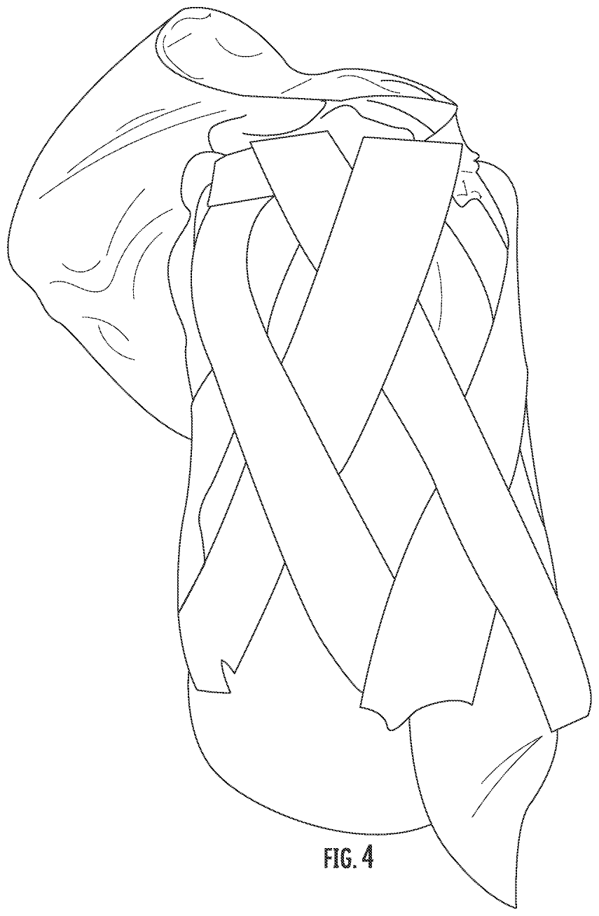

Examples of such layering and/or reinforcement described above are illustrated in FIG. 2 through FIG. 4.

In one embodiment, the natural materials may be prepared in a “prepreg” form. As used herein, the term “prepreg” may be defined as a “pre-impregnated” material that may be saturated in resin and usually kept cooled until needed to mould custom devices. Typically, the prepreg material is a reinforcing fabric that has been pre-impregnated with a resin system that already includes the proper curing agent. Consequently, the prepreg is ready to lay into the mould without the addition of any further resin.

In particular embodiments, when removed from refrigeration the resin may be activated and will set over time. With this method one could use this material to make different braces or orthotics or in conjunction with prosthetic limb production. This method may be used with carbon or other materials in other industries including automotive and aerospace production.

In one embodiment, natural materials such as hemp or natural fibers may be used to produce a media used for additive manufacturing, or 3D printing. In general, an extruder head lays down a filament in discretized layers to create the final product. Accordingly, hemp or natural fibers may be broken down to be used as a printable material for the various prosthetic layers and devices.

In one embodiment, the hemp fiber braided sleeve may include the inclusion of other natural or synthetic materials within the final product.

In further embodiments the bi-directional woven reinforcement strips, may also be embodied as any other type of woven material and may be manufactured without the inclusion of the adhesive backing.

In one embodiment, the prosthetic devices and layers disclosed herein, including at least tri-layered materials and the plant-based resin may be used in conjunction with all other current products used in fabrication of prosthetic and orthotic devices. The products are not exclusive to one another and may be implemented in any existing fabrication process.

In some embodiments, the socket or portion of the prosthesis that fits around and envelopes a limb residuum and to which the prosthetic component or element may be attached is configured to accept a limb residuum (or residual limb or stump), that is, the portion of an arm or leg remaining after an amputation.

In some embodiments, the prosthetic device includes an upper section that is configured to be operatively secured to a socket of an amputee and a lower section that is configured to be operatively secured to a prosthesis. In some embodiments of the prosthetic device, the upper section is configured to be operatively and releasably secured to a socket of an amputee and the lower section is configured to be operatively and releasably secured to a prosthesis. In some embodiments, the prosthetic device includes a prosthetic foot element and/or a prosthetic hand element. In some embodiments, the prosthetic device is operatively and releasably secured to a prosthetic foot element and/or a prosthetic hand element. In some embodiments, the prosthetic device is non-releasably attached to a prosthetic foot element and/or a prosthetic hand element. Thus, in some embodiments, an upper section of the prosthetic device is configured to be operatively and releasably secured or non-releasably secured to a limb residuum. In other embodiments, a lower section of the prosthetic device is configured to be operatively and releasably secured or non-releasably secured to a prosthetic arm and/or hand or leg and/or foot. In still other embodiments, the limb residuum is a portion of an arm or leg. In further embodiments, the prosthetic device includes a prosthetic arm and/or hand element or leg and/or foot element.

In some embodiments, the prosthetic device may include a component that imparts antimicrobial capabilities. In particular, an antimicrobial substance known to be safe to a human body can be used and included in the manufacture of the prosthetic device, for example, as a coating of the final product or components thereof and/or included in the process of forming the resin used in making the prosthetic device, and in some embodiments, this may entail a woven soft sock a patient may wear in-between their limb and a laminated socket forming a part of the prosthetic device system provided herein. The antimicrobial is classified as at least one of an antibacterial, antiviral or antifungal agent. In particular embodiments, the antimicrobial agent includes grepafloxacin, sparfloxacin, clinafloxacin, enoxacin, lemefloxacin, norfloxacin, pipemidic acid, ciprofloxacine, temafloxacine, tosufloxacine, a salt thereof, and a mixture thereof.

Suitable subjects according to the present invention include mammalian subjects. Mammals of the present invention include, but are not limited to, canines, felines, bovines, caprines, equines, ovines, porcines, primates (including non-human primates), humans, and the like. In some embodiments, the mammal is a human subject (living or deceased). Human subjects of both genders are suitable subjects according to the present invention. Further, the subjects relevant to this invention may be male or female and may be any age, but typically at least 3 months old to over 100. Subjects relevant to this invention further include any species and may be of any race or ethnicity, including, but not limited to, Caucasian, African-American, African, Asian, Hispanic, Indian, etc., and combined backgrounds. Particularly relevant subjects to this invention are subjects who are amputees. The subjects may be those human and non-human animals for whom at least a portion of a limb (such as an arm or leg) has been removed.

The present invention can also be carried out on animal subjects, particularly mammalian subjects such as dogs, cats, livestock and horses for veterinary purposes, for screening and development purposes and/or quality of life or necessity purposes.

In addition to the benefits and methods of using the prosthetic devices as described above, embodiments according to the present invention further provide the wearer of the prosthetic device the opportunity to engage in a wide variety of activities which were precluded in the past, or in activities in which the wearer's enjoyment was limited, at least because of the structural limitations and corresponding performance of prior art prostheses. Walking, running, jogging, jumping and other activities are sustained by the prosthetic device and it may be utilized in substantially the same manner as a normal or natural limb of the wearer. The wearer is able to adjust the flexibility of the prosthetic device at his or her discretion.

Embodiments of the present inventive concept also provide materials for products that serve as a substitute for carbon and/or fiberglass materials replacing the industry standard of carbon fiber, fiberglass, or synthetic fiber. These products would then comprise, consist or consist essentially of environmentally friendly, renewable and/or sustainable materials comprising, consisting or consisting essentially of hemp, flax, wood pulp, bamboo, tencel, cotton, wool, camel hair, cashmere, mohair, silk, jute, ramie and sisal and/or other natural fibers.

The materials and methods of layering the same in the fabrication process provides a material with equal or better tensile and integral strength compared to carbon fiber, fiberglass, or synthetic fiber, and in particular, for fabrication and/or use in construction and/or building material, automotive (hoods, grilles, doors, quarter panels, trunks, tailgates, interior parts, roofs, etc.) aircrafts, boats, bicycles (unicycles, tricycles, tandem bicycles, etc.), motorcycles, scooters, hoverboards, and other recreational vehicles, sports equipment, recreational equipment, musical equipment, electronics, ballistics, armor plating and various consumer products, and accessories associated with each. Thus, the present invention provides an alternative source that is a natural material that is a source of a strong, renewable and/or sustainable material. Further, through the use of plant-based or animal-based, natural resources and the bioavailability of these natural materials described herein render the natural fibers a lower cost alternative compared to current standard products (such as carbon, fiberglass, and traditional fabrication materials) used in the construction and product manufacturing industries as well as presents an environmentally friendly option for disposal.

Thus, the inventive concept also discloses products including the natural materials described herein and include, but are not limited to, construction and/or building materials, automotives (hoods, grilles, doors, quarter panels, trunks, tailgates, interior parts, roofs, etc.), aircrafts, boats, bicycles (unicycles, tricycles, tandem bicycles, etc.), motorcycles, scooters, hoverboards, and other recreational vehicles, sports equipment, recreational equipment, musical equipment, electronics, ballistics, armor plating and various consumer products, and accessories associated with each. Methods of making these products are also contemplated herein.

The foregoing embodiments meet the overall objectives of this disclosure as summarized above. However, it will be clearly understood by those skilled in the art that other changes and modifications can be made that are also useful improvements without departing from the scope of the present disclosure, which will now be further described with reference to the following examples. It should be appreciated that these examples are for the purpose of illustrating aspects of the present inventive concept and do not limit the scope of the invention as defined by the claims.

### EXAMPLE

In particular embodiments, the prosthetic device is designed to replicate certain functional aspects of the human leg, especially for lower limb amputees. The prosthetic device is configured to fit over a residual stump of a limb. For example, adaptation is with a socket, which is made to conform to the shape of the stump. In some embodiments, lamination plates having a standard 4 hole threaded therein are used. However, lamination pyramids, rotation adapters, or any means understood by those skilled in the art may be employed. At the lower end, a keel replicates the structure of the foot, and generally has a foot prosthesis of suitable shape and size attached to further imitate the appearance and functional characteristics of a human foot. The shank of the prosthesis is a generally vertical component functioning to transmit forces between the residual limb, or stump, of the amputee and the keel.

In particular methods, in general, fabrication includes taking a cast of the subject's stump and making a socket incorporating the necessary fittings, including an alignment fitting for angular adjustments; tubular fitting for length and rotational adjustment; a fitting for linear adjustments; and an ankle fitting for angular adjustments and to allow attachment of a foot. The area between the top of the keel and the bottom of the socket is foamed in place, and when the foam hardens, the shank is shaped to the desired configuration between the keel and the socket. The prosthesis is prepared for the draping of natural fibers and resin materials as described above until the finished prosthesis is satisfactory.

Further, automated, computer controlled bi-axial and tri-axial braiding of sockets, over a mold or mandrel made of carved foam, plaster or thermoset material in the conventional manner, but also of an easily retrievable and usable wax or water soluble casting material created by a Computer Aided Design (CAD) file on a Numerically Controlled (CNC) machine tool may be employed. The CAD file is created from a 3-D digital image of the subject's residual limb generated by a laser scanner or other sensing mechanism. Braiding also allows incorporation of woven cloth and reinforcements into the braiding process for added strength at selected areas.

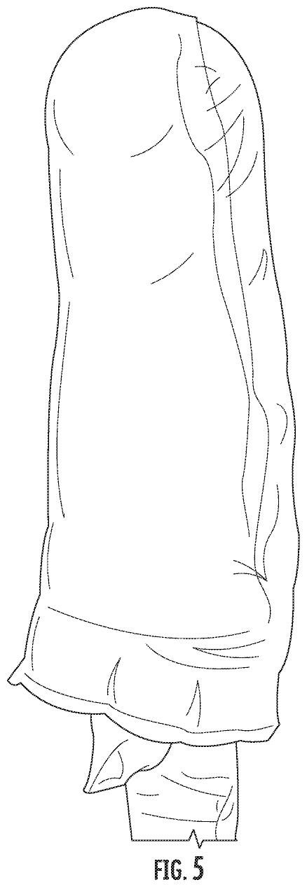

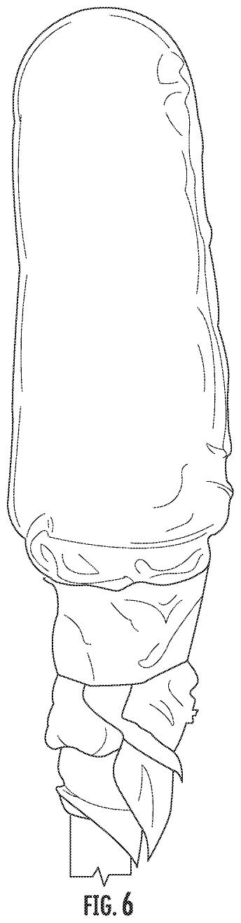

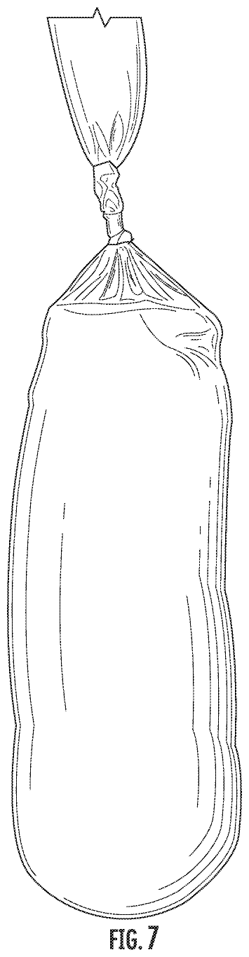

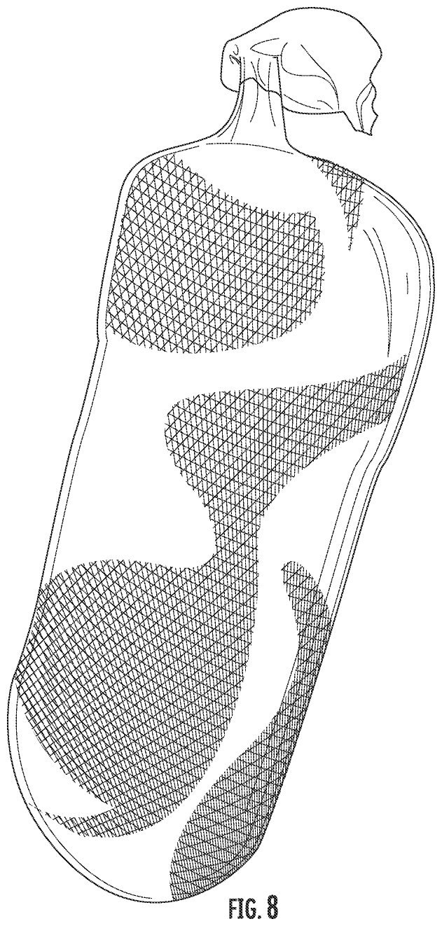

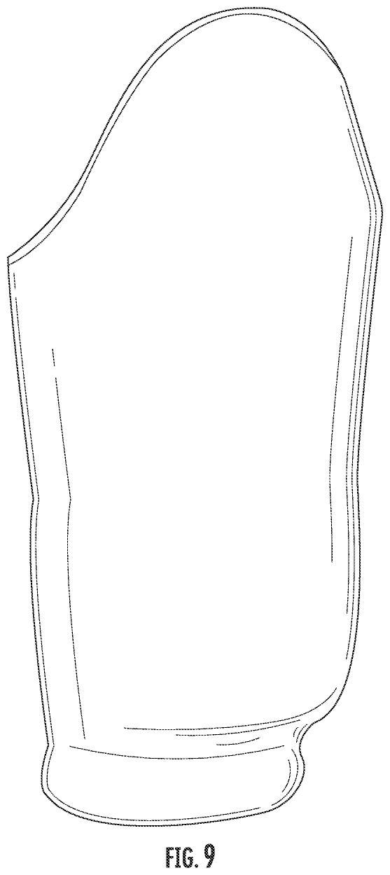

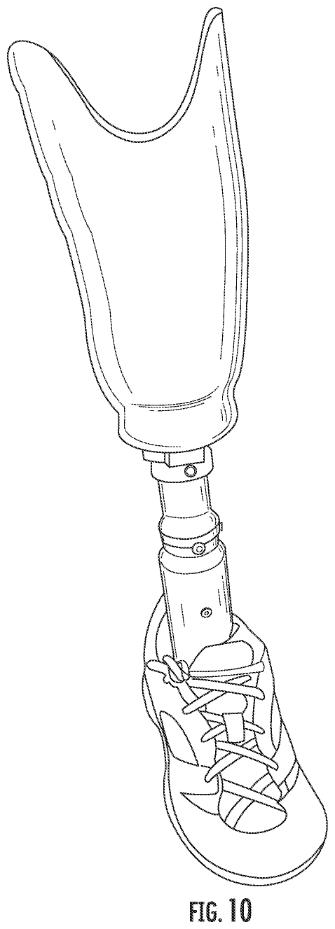

In some specific embodiments, a fiberglass cast of the patient's residual limb is made and then filled with plaster. It may be modified to then blister form a clear plastic “test or check socket.” From there, a practitioner can test fit the patient and heat modify the socket or mark the socket for further modification with further testing if needed for further modifications before making a definitive device. At times a practitioner may have a central fabrication facility provide a test socket and those facilities may use a CNC machine to create a foam model and clear test socket from measurements taken from the patient where such measurements can be taken by hand or with a 3D scanner. Regardless of any method used to obtain a definitive model, the methods and materials of the present invention can be used. See FIG. 2 through FIG. 8.

Moreover, the method of manufacturing the prosthetic device of the present inventive concept further contemplates additive manufacturing, or 3D printing, of the natural fibers used herein to form the limb element of the prosthetic device.

1. A prosthetic device comprising:
a socket configured to accept a limb residuum and consisting essentially of a resin composition reinforced by a material consisting essentially of at least one of a plant-based fiber selected from the group consisting of at least one of hemp, flax, wood pulp, bamboo, and cotton; and
a shank mechanically attached to the socket,
wherein the material is braided to form a braided sleeve and wherein the material does not comprise carbon fiber or fiberglass fibers.

2. The prosthetic device of claim 1, wherein the material is further layered in a manner resulting in a bi-directionally woven strip of natural material.

3. The prosthetic device of claim 1, wherein the plant-based fiber comprises a mat of woven hemp fibers.

4. The prosthetic device of claim 1, wherein the material is bast fibers of hemp.

5. The prosthetic device of claim 1, wherein an upper section of the prosthetic device is configured to be operatively and releasably secured or non-releasably secured to a limb residuum.

6. The prosthetic device of claim 1, wherein a lower section of the prosthetic device is configured to be operatively and releasably secured or non-releasably secured to a prosthetic arm and/or hand or leg and/or foot.

7. The prosthetic device of claim 1, wherein the limb residuum is a portion of an arm or leg.

8. The prosthetic device of claim 1, wherein the device further comprises a prosthetic arm and/or hand element or leg and/or foot element.

9. The prosthetic device of claim 1, wherein the prosthetic device has substantial tensile strength, integral strength and/or is capable of storing and releasing energy.

10. The prosthetic device of claim 1, wherein the socket configured to accept the limb residuum is at least 80% comprised of an environmentally friendly, renewable and/or sustainable material.

11-12. (canceled)

13. A method of manufacturing the prosthetic device of claim 1.

14. The method of claim 13, wherein the method comprises additive manufacturing or 3D printing.

15. The method of claim 13, wherein the plant-based fiber of the prosthetic device is bast fibers of hemp derived from a plant of the Cannabis genus.

16. A socket of a prosthetic device, wherein the socket consists essentially of a resin composition reinforced by a material consisting of bast fibers of hemp derived from a plant of the Cannabis genus, wherein the bast fibers are configured in a braided sleeve and layered to form a bi-directional mat of woven hemp fibers, and wherein the socket does not comprise carbon fiber or fiberglass fibers.

17. (canceled)
